(19) United States
(12) Patent Application Publication
(10) Pub. No.: US 2012/0122052 A1
(43) Pub. Date: May 17, 2012

(54) Medical, in Particular Dental, Diagnostic Device Having Image Capture Means

(75) Inventor: André Hackel, Biberach (DE)

(73) Assignee: Kaltenbach & Voigt GmbH, Biberach (DE)

(21) Appl. No.: 13/291,786

(22) Filed: Nov. 8, 2011

(30) Foreign Application Priority Data
Nov. 11, 2010 (DE) 10 2010 043 794.8

(51) Int. Cl. A61B 6/14

(52) U.S. Cl. 433/29

(57) ABSTRACT

A medical, in particular dental, diagnostic device having an elongate handpiece with means for image acquisition as well as an optical system having at least one optical element for deflecting a light beam that is incident from the side of the handpiece to the means for image acquisition, wherein the angle of deflection of the optical element is smaller than 90°.

The present invention relates to a medical, in particular a dental, diagnostic device according to the precharacterizing clause of claim 1, which device has an elongate handpiece with means for image acquisition, wherein there is further provided an optical system having at least one optical element for deflecting a light beam that is incident from the side of the handpiece to the means for image acquisition. The present invention relates in particular to a diagnostic device for the transillumination of teeth.

In medicine, in particular in dentistry, diagnostic systems based on optical principles are increasingly being used. The reason for this is that such devices usually allow a diagnosis to be made without contact, that is to say in particular in a pain-free manner, and, in addition, often also provide optical images with which any necessary therapeutic measures can be communicated to the patient graphically and hence more clearly. For example, so-called intraoral cameras are used in dentistry, which cameras comprise a handpiece, the front end region of which is introduced into the mouth of a patient. In that end region there is generally a light-entry or viewing window for the camera lens, from which the image of the object to be examined is transmitted to an acquisition device, for example a CCD chip.

Such an intraoral camera can further be extended to a system for the transillumination of teeth, as is known inter alia from DE 10 2006 041 020 A1 of the applicant. In that system, the tooth to be examined is irradiated with light within a specific wavelength range, an optical image of the tooth illuminated by the examination radiation then being acquired and evaluated. Because carious areas in the tooth scatter the light differently than healthy dental tissue, such areas can be identified when the tooth is observed with the aid of a camera, it even being possible, if the system is suitably configured, to obtain a more reliable caries diagnosis than is the case with a conventional X-ray examination.

In particular when molar-tooth surfaces are to be observed with the aid of such a transillumination system, there is limited freedom of movement of the device owing to the risk of collision with the front incisor region. The devices known hitherto conventionally have for image transmission an optical element which effects right-angle beam deflection and transmits the light to the acquisition device, that is to say, for example, the CCD chip. Collision of the hand-held dental device with the other teeth during information recording is the inevitable result of the anatomical conditions. In order to avoid a collision, the device is conventionally positioned at a slight angle, resulting in a non-parallel orientation of the recording device relative to the molar-tooth surface. However, the subsequent comparability and/or reproducibility of the diagnostically recorded values is hindered by the resulting perspective distortion, which is dependent on the recording angle.

Accordingly, the object underlying the present invention is to provide a novel solution in which the above-described disadvantages are avoided.

The object is achieved by a medical diagnostic device having the features of claim 1. Advantageous further developments of the invention are the subject of the dependent claims.

The solution according to the invention is based on the idea of modifying the optical system of the device, which serves to direct the laterally incident light to the image acquisition means, in such a manner that, with the device held at an angle, a parallel recording of the molar-tooth surface is achieved, or the central optical beam path is perpendicular to the object plane. There are used for that purpose one or more optical elements which ultimately effect an image deflection which is smaller than 90°. As a result, a so-called orthograde recording direction relative to the longitudinal direction of the hand-held dental device is achieved, whereby its handling is markedly simplified.

According to the invention there is accordingly proposed a medical, in particular a dental, diagnostic device having an elongate handpiece with means for image acquisition as well as an optical system having at least one optical element for deflecting a light beam that is incident from the side of the handpiece to the means for image acquisition, wherein according to the invention the angle of deflection of the at least one optical element is smaller than 90°.

The optical element for deflection can be in particular a prism arranged in the front end region of the handpiece or a mirror. It would also be conceivable for the optical system to comprise a plurality of elements for beam deflection, which elements altogether effect a deflection of less than 90°. In particular, an angle of deflection of approximately 80° has been found to be particularly advantageous.

A further aspect of the present invention relates to measures for minimizing the height of the part of the device that is introduced into the mouth. To that end, a very short focal length of the optical system is desirable. An enlargement of the image entry angle beyond the limiting value of an optical element for beam deflection that is present in the optical system is then achieved by the additional use of a beam-widening optical element. The beam-widening optical element is preferably located spatially in front of the beam-deflecting optical element in the direction of the object plane, that is to say towards the molar-tooth surface.

According to this further aspect of the present invention there is accordingly proposed a medical, in particular a dental, diagnostic device having an elongate handpiece with means for image acquisition as well as an optical system having at least one optical element for deflecting a light beam that is incident from the side of the handpiece to the means for image acquisition, wherein according to the invention the optical system additionally has an image-widening optical element. As already mentioned above, the image-widening optical element is preferably arranged spatially in front of the element or elements for image deflection. It can in particular be a lens.

The medical diagnostic device according to the invention can in particular be a device which is part of a system for the transillumination of teeth. However, the particular configuration of the optical system is also advantageous if only optical images of tooth surfaces are to be recorded. Consequently, the solution according to the invention can also be used in conventional intraoral cameras.

The invention is to be explained in greater detail hereinbelow with reference to the accompanying drawing, in which:

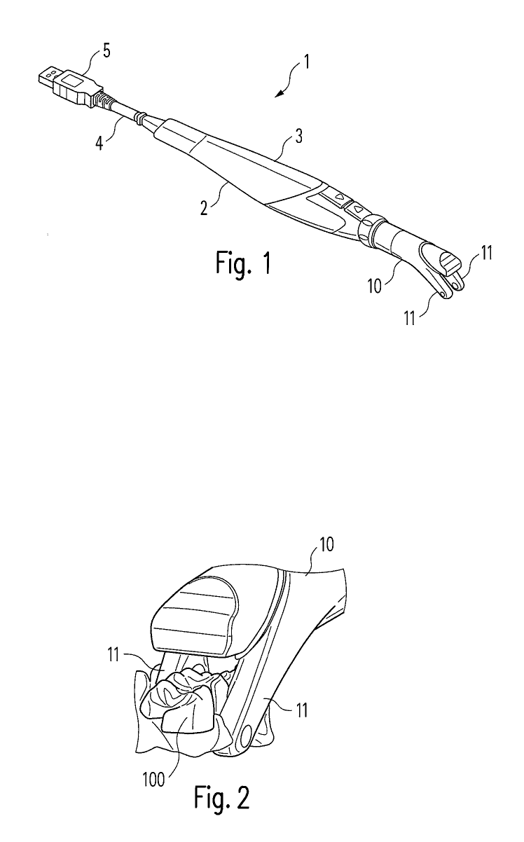

FIG. 1 shows a view of an embodiment according to the invention of a dental diagnostic device;

FIG. 2 shows the front end region of the device of FIG. 1;

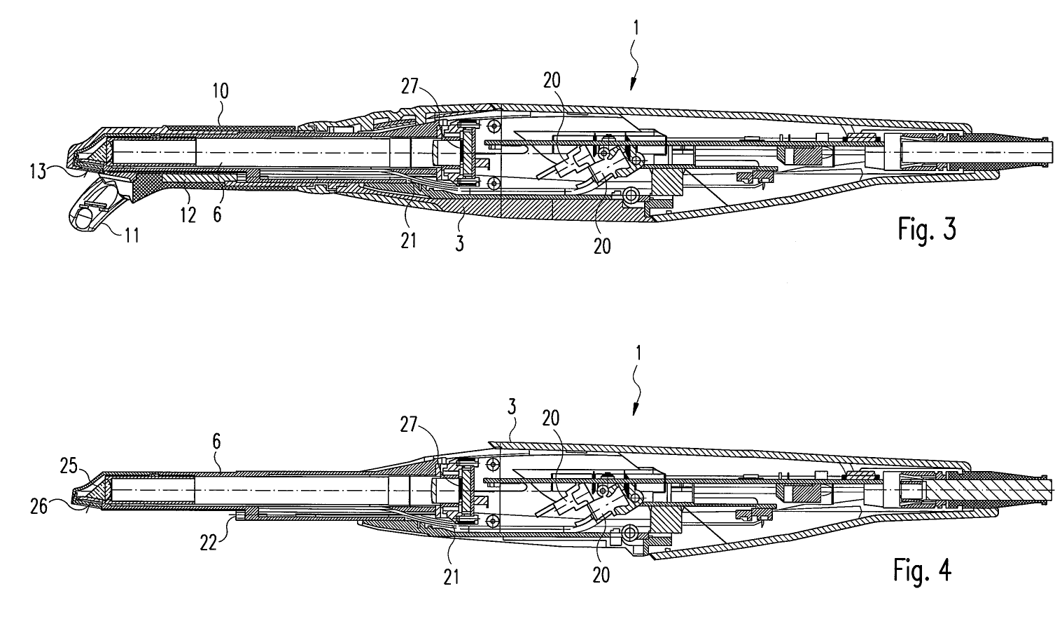

FIGS. 3 and 4 show two sectional views of the diagnostic device;

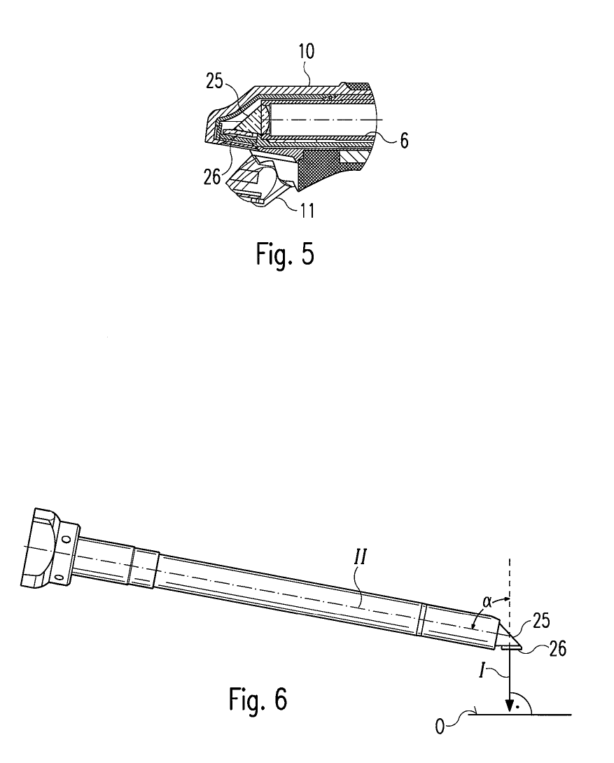

FIG. 5 shows a sectional view of the front end region of the device; and

FIG. 6 shows a schematic view of the means for image transmission configured according to the invention.

As has already been mentioned, the so-called transillumination method represents a preferred application example for the present invention. Its mode of operation will therefore briefly be explained in principle below.

The transillumination method is based on passing visible light through a tooth, that is to say transilluminating it. To that end, examination radiation is generated with the aid of a light source and is directed at the tooth. The examination radiation is usually in a wavelength range of approximately from 550 μm to 790 μm, for example approximately 670 μm. The tissue of the tooth does not completely block the examination radiation but instead allows the light to pass through the tooth. The radiation is thereby partially scattered, carious regions in particular having a characteristic effect on the light. If the tooth transilluminated in that manner is viewed from different viewing directions, such carious regions can be detected because they appear slightly darker. In particular when images taken at different times are compared with one another, caries can thus be detected comparatively effectively and also in good time.

The examination based on the above-described method is carried out according to the invention with the aid of a medical diagnostic device, which is shown in FIGS. 1 and 2.

The device, which is denoted generally by the reference numeral 1, first has an elongate handpiece 2 which contains the fundamental electronic components as well as the optical elements required to carry out the transillumination method. In particular there is arranged inside an elongate handle 3 of the handpiece 2 an image acquisition device, for example in the form of a CCD chip, with the aid of which an optical image of the illuminated tooth is acquired. Furthermore, at least one light source, the light of which is used to transilluminate the tooth, is located inside the handle 3.

The optical image of the tooth acquired with the aid of the image acquisition means is then transmitted to a central unit, for example a PC, for which purpose a cable 4 having a USB connector 5 located thereon is provided on the rear side of the handle 3. Other connection possibilities would be conceivable instead of the USB connector 5, the use of a USB connection having particular advantages because it can at the same time also be used to supply power to the device 1.

Coupling of the light into the tooth 100 to be examined is carried out with the aid of an attachment 10, which is preferably removably arranged at the front end of the handle 3. As is visible in the sectional views of FIGS. 3 and 4, which show the device 1 on the one hand with the attachment 10 fitted to the handle 3 (FIG. 3) and on the other hand without the attachment (FIG. 4), the handle 3 has at its front end a cylindrical pin 6 onto which the attachment 10 can be pushed. At the front end of the attachment 10 there are two lateral arms 11 which rest on the side walls of the tooth 100 to be examined or on the gum, the so-called gingiva.

In the central region of the handle 3 there are arranged one or more light sources 20, from which light guides 21 extend to a front shoulder 22 on the cylindrical pin 6. When the attachment 10 is in the fitted state, the front end regions of the light guides 21 are aligned with corresponding light guides 12 which run inside the attachment 10 and each extend to the front end regions of the arms 11. Here there are located decoupling elements, which decouple the light laterally from the light guides and hence direct it at the side walls or the gingiva of the tooth 100, so that the light emitted by the light sources 20 can effectively be used to illuminate the tooth 100.

A window 13 is further formed in the front end region of the attachment 10, which window forms a light-entry window via which the tooth 100 to be examined can be observed. As is visible in particular in the views of FIGS. 3 and 5, the pin 6 extends into the front end region of the attachment 10, in such a manner that optical means for image deflection are arranged immediately behind the window 13. As will be described in more detail hereinbelow, incident light is deflected with the aid of those optical means, which are formed by a deflecting prism 25 and a lens 26 arranged in front of it, in such a manner that it is directed at a CCD chip 27 located in the handle 3. The chip 27 accordingly records an image of the object located in front of the light-entry window 13.

As already described hereinbefore, it was hitherto conventional to carry out an image deflection by 90°. However, this led to limited freedom of movement because the light-entry window 13 of the attachment 10 could not be oriented perpendicularly to the surface of a molar tooth to be examined because this led to a collision with the incisors. Because, on the other hand, positioning of the device at an angle results in the recording device not being oriented parallel to the tooth surface, there is proposed according to the invention a special configuration of the optical elements for image transmission, with the aid of which the above-described disadvantages are avoided and which is explained in greater detail hereinbelow with reference to FIG. 6.

To this end, FIG. 6 shows primarily the optical components for image transmission, but not the further components of the device according to the invention.

The particular feature of the solution according to the invention is that, with the device held at an angle, a parallel recording of the molar-tooth surface is nevertheless achieved, that is to say the central beam path I is perpendicular to the object surface O. This is achieved by configuring the deflecting prism 25 so that it effects an image deflection of less than 90°. A light beam that is incident parallel to the optical axis I is accordingly deflected by the deflecting prism 25 by an angle α onto the axis II, as is shown in FIG. 6, wherein a is smaller than 90°, preferably approximately 80°. This results in a so-called orthograde recording direction relative to the longitudinal direction of the handpiece 2 corresponding to the axis II. In other words, when the handpiece is positioned at a slight angle in order to avoid a collision with the incisors, optimum orientation towards the surface O to be observed is achieved. The quality of the images thereby obtained, and accordingly the possibility for caries diagnosis, are markedly improved.

It is to be noted that, instead of a single deflecting prism 25, a plurality of optical elements can be arranged one behind the other in such a manner that the sum of the influence of the optical elements permits an orthograde recording direction relative to the longitudinal axis of the hand-held dental device 1. In other words, the beam deflection produced by the totality of the optical elements is again smaller than 90°. In order to improve the reproducibility of the recording, the attachment, or the device, is preferably provided with means which allow the angle of the positioning of the device relative to the molar-tooth surface to be found again. It is thus ensured that recordings are in principle made substantially from the same direction or perspective and are accordingly readily comparable with one another.

In order to minimize the construction height of the region of the device 1 that is introduced into the oral cavity of the patient, a very short focal length of the optical system is further desirable. An enlargement of the image entry angle is achieved by the additional use of a beam-widening optical element, for which purpose the lens 26 arranged in front of the deflecting prism 25 is used. With the aid of this measure which widens the angle of view, the quality of the recordings can additionally be improved, whereby this measure could also expediently be used in the case of an image deflection by 90°, that is to say could be used independently of the particular configuration of the beam deflection described above.

For illuminating the tooth 100 to be examined there is preferably used a narrow-band illumination source in the near-infrared range which delivers light having a wavelength of preferably over 600 nm, in particular in the range 780±30 nm. With the aid of the image acquisition means there is then recorded a scattered-light image whose spectral distribution corresponds to the illumination wavelength. To that end, the image sensor is sensitive in a range of approximately from 400 nm to 1000 nm. The wavelength in the excitation range is relevant for the evaluation, the remainder being superimposed as noise. An element which reduces disturbance variables associated with the surroundings and hence increases the signal-to-noise ratio is preferably integrated into the optical system. Such an element can be an optical high-pass filter for reducing the visible light below the irradiation wavelength. Alternatively, the optical element can also be a band-pass filter, which reduces the component of the light above and below the irradiation wavelength that is acquired by the image acquisition unit. It would be conceivable in particular to produce this optical element by modifying an existing optical element, for example by coating the lens 26 or another lens surface.

Ultimately, therefore, the quality of the images of the tooth to be examined that are obtained is markedly improved by the solution according to the invention because on the one hand the handling of the device is simplified and on the other hand the optical acquisition of the tooth has been optimized. In principle, the measures provided lead to advantages when images are to be produced with the aid of an intraoral camera. Accordingly, the solution according to the invention is not limited to a system for the transillumination of teeth but can in principle also be used in normal intraoral cameras.

1. Medical diagnostic device having an elongate handpiece with means for image acquisition as well as an optical system having at least one optical element for deflecting a light beam that is incident from the side of the handpiece to the means for image acquisition, wherein an angle of deflection of the optical element is smaller than 90°.

2. Medical diagnostic device according to claim 1, wherein the optical element for deflection is a prism arranged in the front end region of the handpiece or a mirror.

3. Medical diagnostic device according to claim 1, wherein the optical system has a plurality of elements for beam deflection, which altogether effect a deflection of less than 90°.

4. Medical diagnostic device according to claim 1, wherein the angle of deflection is approximately 80°.

5. Medical diagnostic device according to claim 1, wherein the optical system additionally has an optical element that widens the angle of view.

6. Medical diagnostic device having an elongate handpiece with means for image acquisition as well as an optical system having at least one optical element for deflecting a light beam that is incident from the side of the handpiece to the means for image acquisition, wherein the optical system additionally has an optical element that widens an angle of view.

7. Medical diagnostic device according to claim 6, wherein the optical element that widens the angle of view is arranged spatially in front of the element or elements for image deflection.

8. Medical diagnostic device according to claim 7, wherein the optical element that widens the angle of view is formed by a lens.

9. Medical diagnostic device according to claim 1, comprising an intraoral camera.

10. Medical diagnostic device according to claim 1, wherein the device is part of a system for the transillumination of teeth.

11. Medical diagnostic device according to claim 5, wherein the optical element that widens the angle of view is arranged spatially in front of the element or elements for image deflection.

12. Medical diagnostic device according to claim 11, wherein the optical element that widens the angle of view is formed by a lens.

13. Medical diagnostic device according to claim 6, comprising an intraoral camera.

14. Medical diagnostic device according to claim 6, wherein the device is part of a system for the transillumination of teeth.
